(19) United States
(12) Patent Application Publication
(10) Pub. No.: US 2021/0187330 A1
(43) Pub. Date: Jun. 24, 2021

(54) APPARATUS AND METHOD FOR ULTRASOUND BEAM SHAPING

(71) Applicant: University of Washington, Seattle, WA (US)

(72) Inventors: Michael R. Bailey, Seattle, WA (US); Adam D. Maxwell, Seattle, WA (US); Akshay Purushottamji Randad, Seattle, WA (US); Mohamed Abdalla Ghanem, Seattle, WA (US)

(73) Assignee: University of Washington, Seattle, WA (US)

(21) Appl. No.: 17/268,838

(22) PCT Filed: Aug. 14, 2019
(86) PCT No.: PCT/US2019/046501

§ 371 Date: Feb. 16, 2021

Related U.S. Application Data
(60) Provisional application No. 62/765,101, filed on Aug. 16, 2018.

(51) Int. Cl. A61N 7/00; G16H 20/40

(52) U.S. Cl. CPC A61N 7/00; A61B 17/225; G16H 20/40

(57) ABSTRACT

Apparatus and method for ultrasound beam shaping are disclosed herein. In one embodiment, an ultrasonic therapy system is configured to apply ultrasound to a target in a body. The system includes: an ultrasonic transducer configured to generate the ultrasound; and a customizable lens configured to focus the ultrasound onto a focal area of the target. The target is an object or a portion of the object in the body. The customizable lens is designed and produced based on at least one acquired image of the target.

### STATEMENT OF GOVERNMENT LICENSE RIGHTS

This invention was made with government support under Grant Nos. K01 DK104854 and P01 DK043881, awarded by the National Institutes of Health. The government has certain rights in the invention.

### BACKGROUND

Ultrasound has been employed to diagnose and facilitate removal of soft tissues such as tumors or calcifications such as kidney stones in the body. Ultrasound can be used to ablate soft tissue by thermal or mechanical means. Ultrasound can also be used to noninvasively image stones, manipulate them with radiation force, or fragment them to small pieces so that they can be passed easier.

FIG. 1A is a side plan view of an ultrasound system 10 in accordance with conventional technology. The ultrasound system 10 includes a housing 30 that is filled with a medium 32 (typically water) that facilitates transmission of the ultrasound within the system 10. The ultrasound system 10 includes a test target 22 representing, for example, a hypothetical calcification or tumor in a body. The target 22 is held by a target holder 24.

A transducer 12 generates vibrations at ultrasound frequencies (e.g., from about 20 kHz to about 10 MHz). The transducer 12 can be a piezoelectric element that expands and shrinks with changing electrical polarity applied to the transducer. Such a change in electrical polarity can be applied by an alternating current (AC) at a target ultrasound frequency. An interface 14 permanently attaches a lens to the transducer 14. The interface 14 is typically a permanent epoxy or other suitable strong adhesive. In operation, the lens 16 focuses the ultrasound generated by the transducer 12 through an acoustic window 18 onto a target 22. Much like optical systems, acoustic waves obey Snell's law. For that reason, ultrasound can be shaped by the lens 16 in the path of a propagating acoustic wave. Acoustic lenses (refractive lenses) bend the propagating wave in proportion to the ratio of indices of refraction of the lens and of a target medium, such as biological tissue. The index of refraction is a material property, depending on the speed of sound in the material.

A coupling 17 (e.g., gel, oil, etc.) provides acoustic coupling for the ultrasound propagating toward the target 22. The ultrasound system 10 includes an absorber 26 that prevents ultrasound from escaping into the environment. The operation of the transducer 12 can be controlled by a controller 40.

FIG. 1B is a side plan view of an ultrasound system 10 in accordance with conventional technology. The illustrated conventional system 10 does not include the lens. Instead, the transducer 12 has a shaped surface 12-1 that focuses the ultrasound onto the target 22.

FIGS. 2A and 2B are isometric views of ultrasound transducers with lenses in accordance with conventional technology. The lens in FIG. 2A is a Fresnel lens 16, and the lens in FIG. 2B is a spherical lens. Either of these conventional lenses produces a respective target pressure of the ultrasound by focusing the ultrasound beam to a predetermined focal area. However, one of the challenges in developing ultrasound instruments for moving and breaking urinary tract stones is generating the appropriate acoustic pressure and beam shape to effectively apply force to or fragment the stone. For example, if a beam is too narrow, it may not fragment a stone because it does not impart enough energy on the entire stone. On the one hand, if a beam is too wide ultrasound energy is wasted on collateral tissue. Therefore, focusing of ultrasound is often necessary to achieve sufficient pressure on the stone. However, the focal area in a spherically focused beam is dictated by some beam parameters, such as the transducer frequency, acoustic aperture, and focal length. In practical applications, these parameters are constrained by the size of acoustic window, the depth of target, and pressure and frequency needed to achieve the target effect on the stone.

With soft tissue ablation, a similar challenge exists. For example, high frequency and highly focused transducers achieve precise targeting, but also end up having a relatively small focus area, which slows down treatment. On the other hand, low frequency transducers typically lack targeting precision, and may create unpredictable cavitation of the target and/or the surrounding tissue. This shortcoming of the conventional technology is described with reference to FIGS. 3A-3C below.

FIGS. 3A-3C are schematic views of targeting objects in accordance with conventional technology. In each Figure, the combination of the transducer 12 and the lens 16 produces a focal zone 20 at a given distance from the transducer and for a given frequency of the transducer. However, in practical situations, size and shape of the target 22a-22c (for example, a calcification in human or animal body) is not fixed. As a result, the focal zone 20 is too large for the target 22a and too small for the target 22c, while not having a proper outline for the target 22b.

Accordingly, there remains a need for ultrasound treatment systems that optimize treatment time and consistency by controlling geometry and volume of ablation for different applications.

### SUMMARY

This summary is provided to introduce a selection of concepts in a simplified form that are further described below in the Detailed Description. This summary is not intended to identify key features of the claimed subject matter.

Briefly, the inventive technology is directed to generating an ultrasound beam to treat objects in a body (e.g., stones, calcifications, tumors, etc.). Some examples of such treatment are tissue ablation, lithotripsy, repositioning of stones or stone fragments, etc.

In different embodiments, the shape and volume of the ultrasound treatment area is controlled by a customizable lens and/or a phased array ultrasound transducer. The customizable lens may be designed using the iterative angular spectrum approach (IASA). In some embodiments, the customizable lens is an acoustic diffractive lens (also referred to as “lens,” “customizable lens,” “diffractive lens,” or “holographic lens”) that generates phase offsets and redirection in the wave front as the ultrasound waves transit the lens.

When the customizable lens is mated to an ultrasound transducer having a prescribed amplitude and frequency of the ultrasound, the customizable lens develops a pattern of phase that, in turn generates a target beam pattern of pressure amplitude and phase at the target focal surface. Furthermore, in some embodiments the amplitude/phase distribution of the ultrasound may be controllable in several different planes of ultrasound propagation to produce different patterns of ultrasound pressure amplitudes/phases at these target planes. In some embodiments, the target treatment areas are selectable by adjusting the frequencies of the transmitted ultrasound. In some embodiments, the customizable lens is produced by three dimensional (3D) additive printing. The customizable lens may be attached-to and removed-off the transducer with a quick-change holding mechanism and a temporary interface.

In some embodiments, the ultrasound beam creates amplitude/pressure fields that apply radiation force in a desired direction. For example, the ultrasound beam may create a 2-dimensional or 3-dimensional potential well around an object to trap it in a position. In other embodiments, the amplitude/pressure field can have a phase gradient imposed, therefore moving a stone or other solid object along a gradient in a predetermined path. Alternatively, a stone or other object may be blocked from moving down a path or into a certain area by a beam that forms a barrier based on the ultrasound amplitude or phase gradient.

In one embodiment, an ultrasonic therapy system configured to apply ultrasound to a target in a body includes: an ultrasonic transducer configured to generate the ultrasound; and a customizable lens configured to focus the ultrasound onto a focal area of a target. The target is an object or a portion of the object in the body, and the customizable lens is designed and produced based on at least one target.

In one aspect, the target is defined by at least one acquired image of the object in the body. In another aspect, the customizable lens is a three-dimensional (3D) printed lens. In another aspect, the customizable lens is designed based on an iterative angular spectrum approach (IASA).

In one aspect, the focal area corresponds-to or exceeds a size and a shape of the object. In another aspect, the ultrasound pressure amplitudes are focused onto the focal area. In one aspect, the ultrasound pressure phases are focused onto the focal area.

In one aspect, the customizable lens is configured to produce a plurality of target distributions of the ultrasound at a corresponding plurality of focal distances from the customizable lens. In another aspect, the customizable lens is configured to produce the plurality of target distributions of the ultrasound at a corresponding plurality of ultrasound frequencies.

In one aspect, the at least one acquired image of the object is modified to introduce an asymmetry in a target ultrasound field of the focal area of the object. In another aspect, the customizable lens produces multiple high-pressure areas within the target ultrasound field.

In one aspect, the system also includes: a mechanism configured to mate the customizable lens with the ultrasonic transducer; and an interface material configured to temporarily attach the customizable lens with the ultrasonic transducer. In one aspect, the mechanism is selected from a group consisting of a quick-change clamp, a hinge and a bolt.

In one aspect, the ultrasound transducer is a phased array transducer comprising a plurality of ultrasound sources. In another aspect, the plurality of ultrasound sources of the phased array transducer is arranged along a curved surface.

In one embodiment, a method for applying an ultrasound to a target in a body includes: defining a customizable lens based on the target, wherein the target is an object or a portion of the object in the body; generating the ultrasound by an ultrasonic transducer; and focusing the ultrasound onto a focal area of the object by the customizable lens.

In one aspect, the method includes acquiring an image of the object, wherein the customizable lens is defined at least in part based on the image of the object. In one aspect, the method also includes: acquiring additional images of the object while the target is being treated; and based on acquiring the additional images of the object, modifying the customizable lens. In one aspect, the method also includes manufacturing the customizable lens by three-dimensional (3D) additive-printing. In one aspect, the method also includes: applying an interface material to a surface of the customizable lens; mating the customizable lens with the ultrasonic transducer via the interface material; and, after focusing the ultrasound onto the focal area, removing the customizable lens from the ultrasonic transducer.

In one aspect, the ultrasound transducer is a phased array transducer comprising a plurality of ultrasound sources. In another aspect, focusing the ultrasound onto the focal area of the object includes focusing ultrasound pressure amplitude distribution or ultrasound pressure phase distribution over the focal area.

In one aspect, the method also includes: prior to defining the customizable lens, introducing an asymmetry in a target ultrasound field of the focal area of the object; and, in response to introducing the asymmetry, generating multiple high-pressure areas within the target ultrasound field by focusing the ultrasound onto the focal area by the customizable lens. In one aspect, the method also includes generating a plurality of target distributions of the ultrasound at a corresponding plurality of focal distances from the customizable lens.

In one embodiments, a non-transitory computer readable medium having computer executable instructions stored thereon that, in response to execution by one or more processors of one or more computing devices, cause the one or more computing device to perform actions including: acquiring an image of an object in a body; and determining a shape of a customizable lens based on an acquired image of the object in the body, where the customizable lens is configured for mating with an ultrasound transducer, and where the customizable lens is configured to focus the ultrasound transducer onto a focal area at the object.

### DESCRIPTION OF THE DRAWINGS

The foregoing aspects and many of the attendant advantages of the inventive technology will become more readily appreciated as the same are understood with reference to the following detailed description, when taken in conjunction with the accompanying drawings, wherein:

FIGS. 1A and 1B are side plan views of ultrasound systems in accordance with conventional technology;

FIG. 2A is an isometric view of an ultrasound transducer having a Fresnel lens in accordance with conventional technology;

FIG. 2B is an isometric view of an ultrasound transducer having a spherical lens in accordance with conventional technology;

FIGS. 3A-3C are schematic views of targeting objects in accordance with conventional technology;

FIG. 4 is a graph of a target object in accordance with an embodiment of the present technology;

FIG. 5 is a schematic diagram of a method for designing a customizable lens or a phased array in accordance with an embodiment of the present technology;

FIGS. 6A and 6B are graphs of source phase and lens thickness, respectively, for a lens in accordance with an embodiment of the present technology;

FIG. 6C is a graph of pressure amplitude produced by the lens in accordance with an embodiment of the present technology;

FIG. 7A is an isometric view of the lens in accordance with an embodiment of the present technology;

FIG. 7B is a cross-sectional view of the lens of FIG. 7A;

FIGS. 8A and 8B are respectively graphs of target image and pressure amplitude for a target at 10 mm distance from a source of ultrasound in accordance with an embodiment of the present technology;

FIGS. 9A and 9B are respectively graphs of target image and pressure amplitude for a target at 30 mm distance from a source of ultrasound in accordance with an embodiment of the present technology;

FIGS. 10A and 10B are respectively graphs of target image and pressure amplitude for a target at 45 mm distance from a source of ultrasound in accordance with an embodiment of the present technology;

FIG. 11 is a graph of source phase for a lens in accordance with an embodiment of the present technology;

FIG. 12 is a graph of lens thickness for the lens of FIG. 11;

FIG. 13 is a schematic diagram of using the lens in accordance with an embodiment of the present technology;

FIG. 14A is a graph of a target image at 10 mm distance from a source of ultrasound in accordance with an embodiment of the present technology;

FIG. 14B is a graph of pressure amplitudes for the target image of FIG. 14A;

FIG. 15A is a graph of a target phase at 10 mm distance from a source of ultrasound in accordance with an embodiment of the present technology;

FIG. 15B is a graph of phase distribution for the target phase of FIG. 15A;

FIG. 16A is a graph of a target image at 25 mm distance from a source of ultrasound in accordance with an embodiment of the present technology;

FIG. 16B is a graph of pressure amplitudes for the target image of FIG. 16A;

FIG. 17 is a graph of source phase for a lens in accordance with an embodiment of the present technology;

FIG. 18 is a graph of lens thickness for the lens of FIG. 16;

FIG. 19 is a schematic diagram of a phased array in accordance with an embodiment of the present technology;

FIGS. 20A and 20B are graphs of sample pressure fields in accordance with embodiments of the present technology;

FIGS. 21A-21D are graphs of sample pressure fields at different focal distances in accordance with embodiments of the present technology;

FIGS. 22A and 22B are graphs of sample pressure fields in accordance with embodiments of the present technology; and

FIGS. 23A and 23B are schematic drawings of a lens holding mechanism in accordance with embodiments of the present technology.

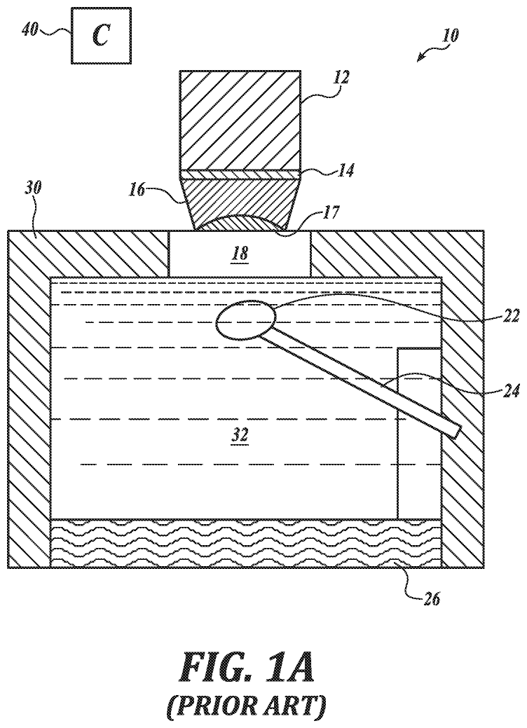

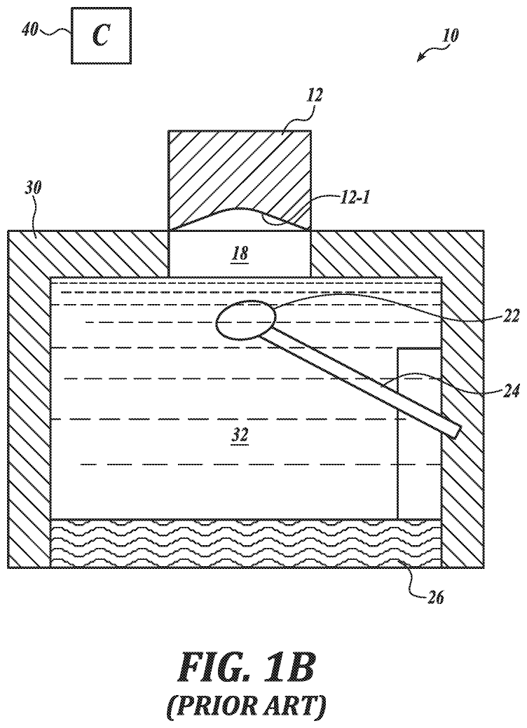

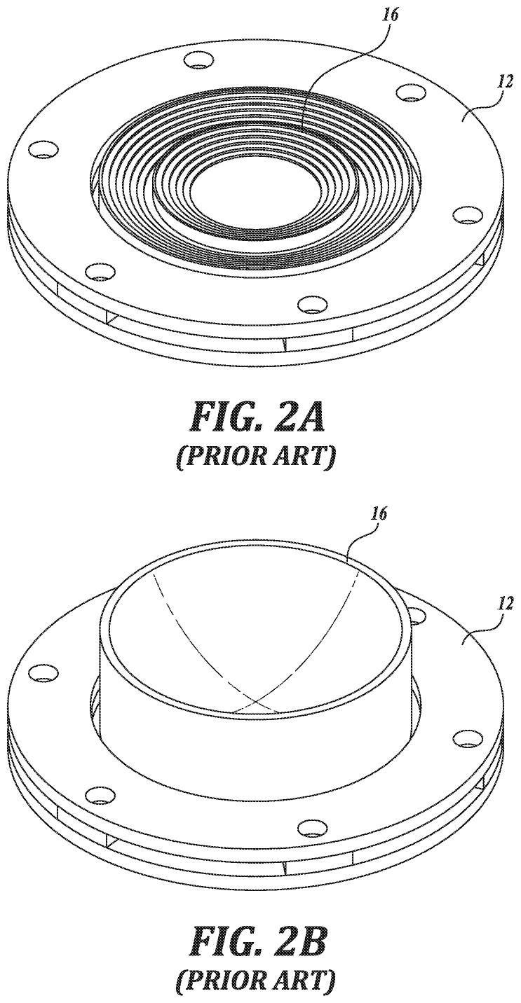

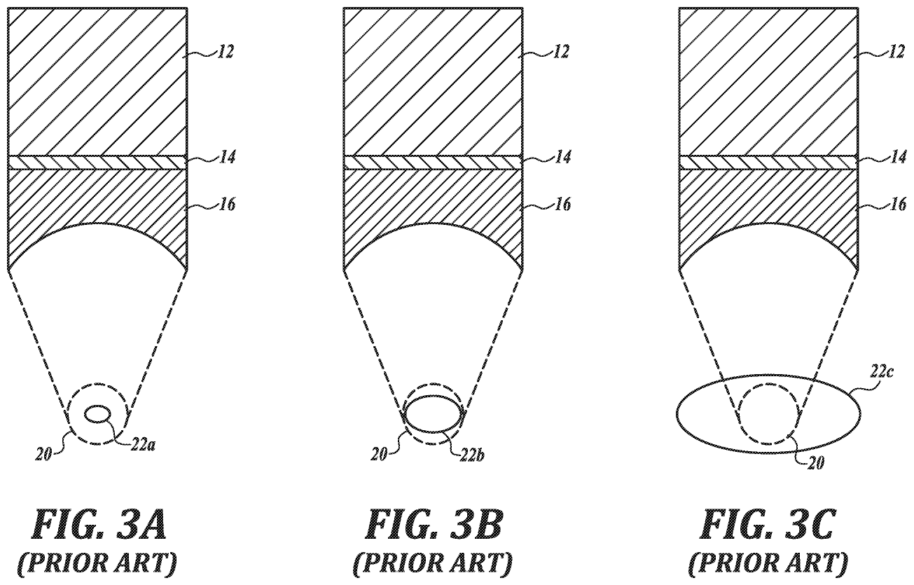

### DETAILED DESCRIPTION

While several embodiments have been illustrated and described, it will be appreciated that various changes can be made therein without departing from the spirit and scope of the claimed subject matter.

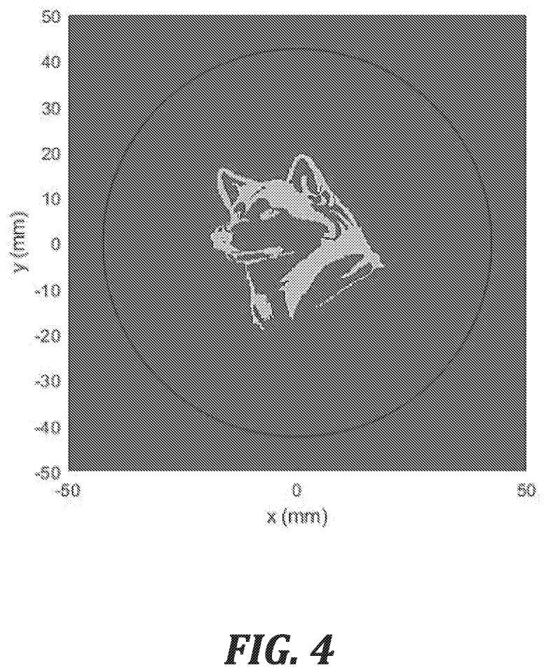

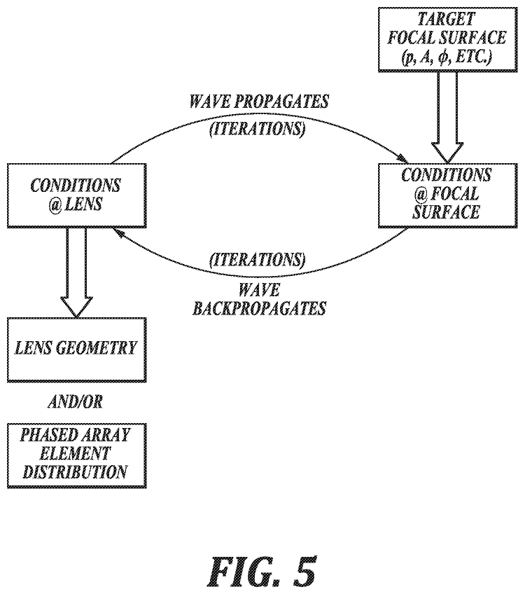

FIG. 4 is a graph of a target object in accordance with an embodiment of the present technology. The horizontal and vertical axes represent the target image in millimeters. The sample target image is 32 mm away from the source of the ultrasound. The target image is binary, the bright areas representing the maximum target pressure and dark areas representing zero target pressure. However, in different embodiments non-binary distributions of target pressure amplitude or phase may be used. The white circle shows the outline of the transducer aperture (which is in the 0 mm plane). With the illustrated embodiment, the transducer and lens attempt to re-create the illustrated pressure field at 32 mm distance from the source of the ultrasound. A method of designing such customizable lens is described with reference to FIG. 5 below.

FIG. 5 is a schematic diagram of a method for designing a customizable lens or an ultrasound phased array in accordance with an embodiment of the present technology. In operations, the lens (also referred to as “customizable lens,” “diffractive lens,” or “holographic lens”) stimulates additive and destructive interference in a propagating wave front to generate a desired pressure and/or phase pattern at a target focal surface. The iterative angular spectrum approach (IASA) develops precise phase mappings for the lenses, which in turn provide a physical design for the lens geometry. As explained with reference to the conventional technology, typical approaches to lens design, such as the Fresnel approximation, fail to produce the desired pressure pattern with sufficient precision when the feature size in desired pressure pattern approaches the wavelength of the propagating wave front.

In some embodiments, the customizable lens may be designed using the iterative angular spectrum approach (IASA). The method is described with reference to designing a customizable lens, but the method can also determine distribution and operation of the elements of an ultrasonic phased array transducer.

In some embodiments, an algorithm implements IASA numerically by iteratively comparing simulated conditions at the target focal surface against the target conditions at the focal surface. In some embodiments, an algorithm implements IASA numerically by iteratively comparing simulated conditions at the focal surface against the target conditions at the focal surface; and the complex pressure distribution at the source to the results from the previous iterative step.

In a first step, the algorithm introduces lens geometry, propagating wave front, and target focal surface in a given medium. The target focal surface may be defined by its pressure pattern (p), made up of an amplitude map (A) and a phase map (Φ). The target focal surface is located some known distance from the lens.

The pressure wave equation includes amplitude and phase functions describing pressure at a given position in Euclidean space:

p(x,y,z)=\hat{p}(x,y,z)e^{jΔΦ(x,y,z)} (1)

where \hat{p}(x, y, z) and ΔΦ(x, y, z) are the amplitude and phase functions, respectively.

The IASA method uses fast Fourier transform (FFT) and inverse fast Fourier transform (IFFT) methods to converge to an optimum error criterion, calculated as an error between the target focal surface and conditions at the focal surface. The general form of the FFT equation in Euclidean space is shown in Equation 2:

P(kₓ,k_{y})=∫∫_{-∞}^{+∞}p(x,y,0)e^{-j(k}^{ₓ}^{x+k}^{_{y}}^{y)}dxdy (2)

The output of the FFT equation, P(kₓ,k_{y}), gives an angular spectrum, where kᵢ is the wavenumber in i space. The IFFT equation, excluding the evanescent wave components, is shown in Equation 3:

$$p ( x , y , z ) = \frac{1}{4 {π}^{2}} \int {\int }_{{k}_{x}^{2} + {k}_{y}^{2} \leq {k}^{2}} P ( {k}_{x} {k}_{y} ) {e}^{j ( {k}_{x} x + {k}_{y} y + \sqrt{{k}^{2} - {k}_{x}^{2} - {k}_{y}^{2}} z )} d {k}_{x} d {k}_{y} \qquad ( 3 )$$

which provides the conditions at the focal surface in Euclidean space from the angular spectrum (P).

In the initial iteration of the loop shown in FIG. 5, the propagating wave front is transformed by FFT into an angular spectrum. A propagation function, shown in Equation 4, then calculates the effect of movement through a given medium on the angular spectrum:

P(kₓ,kᵤ,z)=P(kₓ,k_{y},0)e^{jz}\sqrt{k²−kₓ²−k_{y}²} (4)

which is used to calculate both propagation and backpropagation through the given medium between the focal surface and the lens. The propagating wave front then propagates through the lens and the given medium to produce an angular spectrum for a propagated wave front at the focal surface (the conditions at the focal surface).

As shown in FIG. 5, IFFT provides a wave equation in spatial coordinates for comparison to the desired conditions at the target focal surface. The error criterion indicates whether the lens design at the current iteration produces the target focal surface. In initial iterations, the error between the conditions at the focal surface and the target focal surface may be significant, due to near field effects that impact the propagating wave front.

To account for the near field effects, the IASA incorporates a back-propagation of the propagated wave front from the focal surface to the lens, shown as a clockwise lower arrow in FIG. 5, and modulates the propagating wave front, and its angular spectrum, for iterative propagation forward to the focal surface. The algorithm retains the latest iteration of the phase information at the focal surface to calculate convergence.

In addition to conventional IASA method, the method uses the multiple checks in the convergence criterion to meet our desired goals. The algorithm iteratively compares the convergence of the simulated conditions to the target image specified at each target location. Second, after the first iteration step and in parallel to the previous check, the algorithm compares the complex pressure distribution at the source to that of the previous step as well to speed up and improve the convergence calculation criterion. The comparisons in the previous two checks are specified to be within a specific error tolerance below which convergence to the optimal hologram solution is achieved. Finally, a maximum number of iterations is determined for each run, such that when it is exceeded the method terminates and saves the optimal hologram solution. The error tolerance and maximum number of iterations is determined based on the complexity of the hologram, such as, the number of target locations for phase and or amplitude at different frequencies. These checks of convergence are checked at each iteration step to yield the optimal solution.

Incorporating back propagation into an iterated forward propagating wave equation permits a more precise calculation of the conditions at the focal surface for subsequent adjustment of the lens geometry. With each cycle of forward propagation and back propagation the conditions at the focal surface and the conditions at the lens converge to an optimal solution.

An output of the IASA algorithm is the lens geometry. As described in Equation 5, a spatial thickness parameter describes the lens geometry by taking into account the transmission coefficient (α) of the system, including acoustic impedances (Z) of the lens material (h), the given medium (m), and a transducer (t), a source of acoustic waves:

$${α}_{T} ( x , y ) = \frac{4 {Z}_{t} {Z}_{h}^{2} {Z}_{m}}{{{z}_{h}^{2} ( {Z}_{t} + {Z}_{m} )}^{2} cos {( {k}_{h} T ( x , y ) )}^{2} + {( {Z}_{h}^{2} + {Z}_{t} {Z}_{m} )}^{2} sin {( {k}_{h} T ( x , y ) )}^{2}} \qquad ( 5 )$$

The thickness of the lens (T) can be calculated from the angular spectrum of the converged solution by creating a phase map for the surface of the lens. The lens creates constructive and destructive interference in the near-field by introducing phase offsets (ΔΦ) in the propagating wave front as it passes through the holographic lens. The thickness of the lens is described as follows in Equations 6-7:

Δω(x,y)=(kₘ−kₕ)ΔT(x,y) (6)

where T(x,y)=T₀−ΔT(x,y). (7)

The IASA algorithm is capable of designing a lens that produces multiple target focal surfaces at as many distances from the lens in a given medium. To accomplish this, the IASA algorithm separately incorporates the backpropagation from the wave equations of each of the target focal surfaces when modulating the propagating wave equation.

In a similar manner, the Euclidean coordinate space of the solution permits a phased array element distribution to produce one or more target focal surfaces, by calculating IASA converged solutions for multiple propagating waves from an array of transducers. The IASA method can be used with different transducer geometries. For instance, for a focused transducer, the exact pressure field can be simulated and verified through holographic scanning in a plane. Next, the pressure field at the transducer aperture (obtained by back-projection) is used as the initial boundary condition over which we can impose the required phase to obtain the desired beam shape.

When compared to the conventional lens design methods, the IASA-based design method maximizes the power of the beam while producing an arbitrary pressure distribution in the plane of interest. Furthermore, the method can be extended to constrain the amplitude distribution in several different planes of propagation. Analogously, the method can be extended to produce different beam patterns using ultrasound transducers at different frequencies. The method can also be used to constrain the phase distribution in one or more planes, or both amplitude and phase distributions simultaneously. The desired target field may be binary or continuously varying in amplitude and/or phase over the focal plane of interest.

The IASA method can also be used with different transducer geometries. For instance, for a focused transducer, the exact pressure field can be simulated and verified through holographic scanning in a plane. Then the pressure field at the transducer aperture (obtained by back-projection) is used as the initial boundary condition over which we can impose the required phase to obtain the desired beam shape.

The above-described method for defining the thickness and shape of the lens uses the IASA. However, in different embodiments, other iterative methods for defining the thickness and shape of the lens are also possible. Sample results obtained with the IASA-designed lenses are described below.

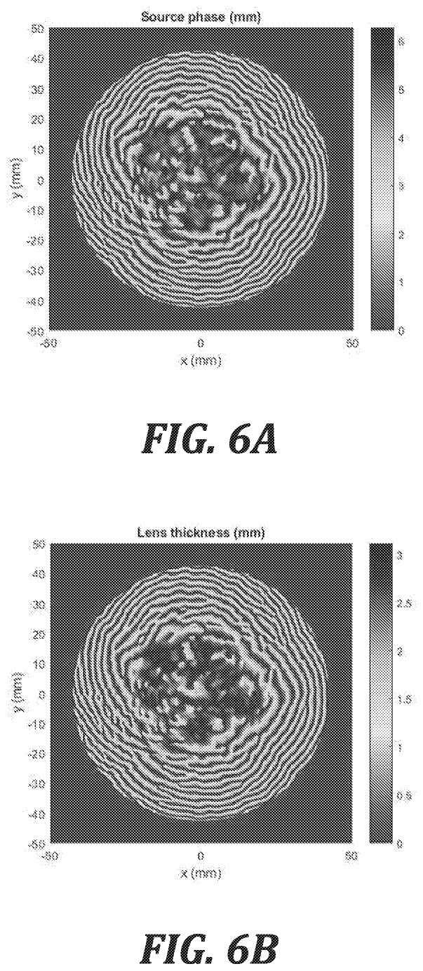

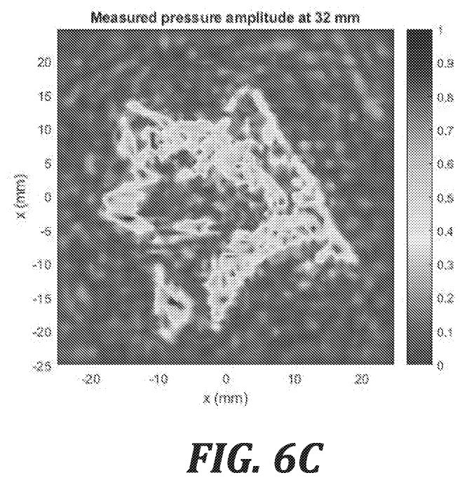

FIG. 6A is a graph of source phase for a lens in accordance with an embodiment of the present technology. FIG. 6B is a graph of lens thickness for that lens. As a result of the modeling described with reference to FIG. 5, the thickness of the customizable lens (diffractive lens) is determined across the surface of the lens. When the customizable lens is attached to the ultrasound transducer to follow oscillations by the transducer, the local phases of the ultrasound at the surface of the customizable lens are distributed as shown in FIG. 6A. These phase offsets constructively/destructively combine to generate a desired pressure field at the target focal distance.

In the illustrated embodiment, the pressure amplitude field (i.e., distribution) that is generated at 32 mm distance from the source is shown in FIG. 6C. As seen by comparing this measured pressure amplitude field (also referred to as a “hologram”) in FIG. 6C with the target pressure field of FIG. 4, the customizable lens produces a good match against the target pressure field. This sample target pressure field has a relatively complex shape in comparison to a real target object in a body. Therefore, the method appears capable of focusing ultrasound pressure over the target object in a body.

In some embodiments, the customizable lens produces phase patterns, whereby the phase of the propagating wave front varies with position on the focal surface. This phase front of the propagating wave may permit non-invasive repositioning of a target located on a focal surface. Such targets may include kidney stones, bladder stones, calcifications, and other endogenous materials lodged in an anatomical vessel. In some embodiments, the customizable lenses create a pressure well around the target, pushing the target toward an area of relative low pressure.

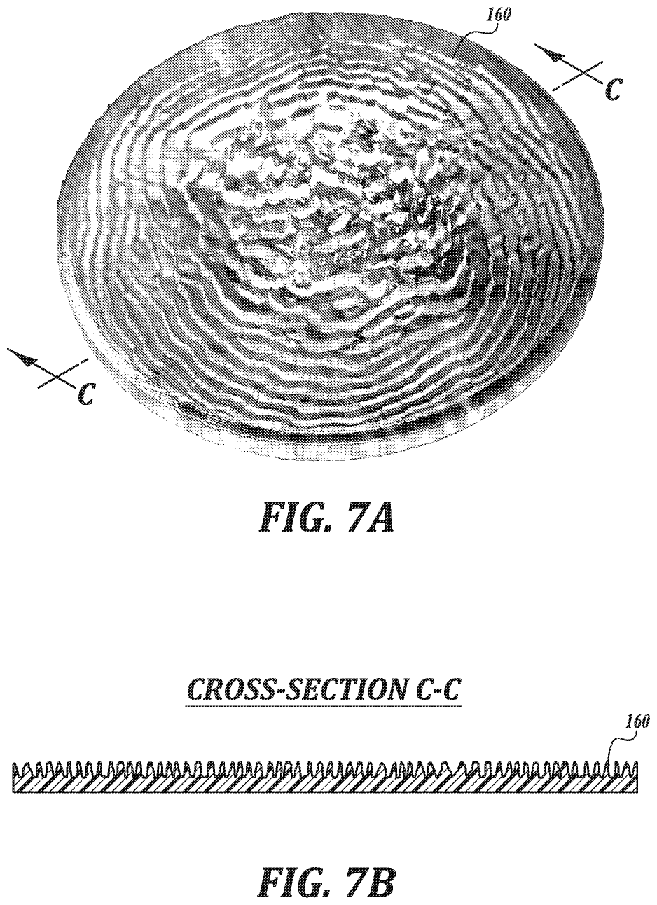

FIG. 7A is an isometric view of a customizable lens 160 in accordance with an embodiment of the present technology. When mated with a transducer that operates at the design frequency of the ultrasound, the customizable lens 160 generates a pressure distribution shown in FIG. 6C. In some embodiments, the customizable lens 160 is made by machining or by additive manufacturing processes, for example, by 3D printing. The customizable lens 160 may be manufactured from glass or plastic that has suitable transmission coefficients for the ultrasound frequency. In some embodiments, the customizable lens 160 may have a curvature to achieve focusing or defocusing simultaneously with image formation (e.g., formation of the target pressure field or phase distribution). The lens resolution is generally determined by the method of manufacture, but in some applications the lens resolution can be smaller than a wavelength.

FIG. 7B is a cross-sectional view of the customizable lens of FIG. 7A. The flat side of the customizable lens 160 mates with the transducer 12, and the non-uniform side of the customizable lens faces the object that is treated by the ultrasound. In some embodiments, the customizable lens 160 may be curved. In operation, the small-scale features on the surface of the lens determine the phase offsets of the emitted ultrasound. When the ultrasound is generated at the required ultrasound frequency, these phase offsets constructively/destructively combine into a target pressure and/or phase field. As explained above, the thickness of the customizable lens 160, that is the size and distribution of the features of the customizable lens, is determined using the IASA method.

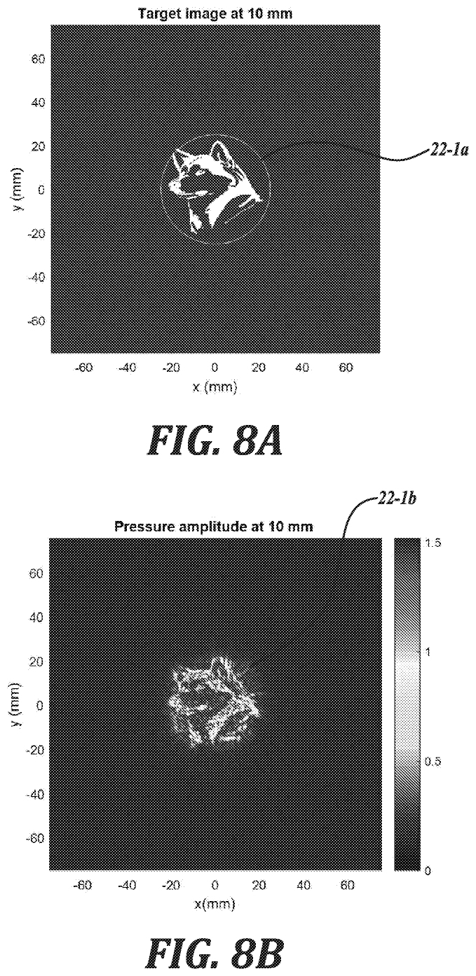

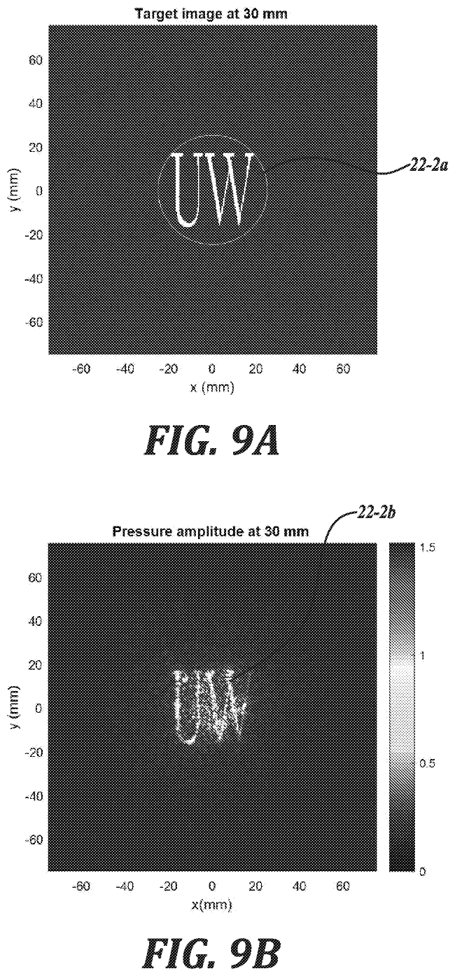

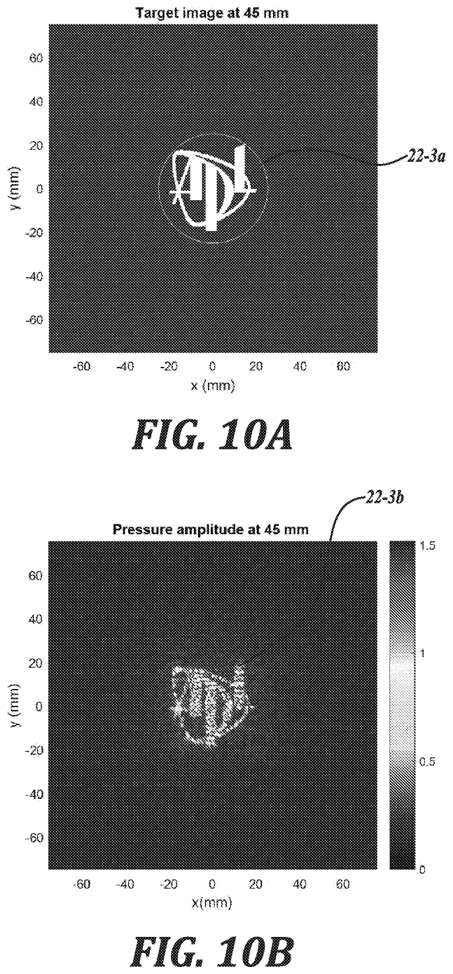

FIGS. 8A, 9A and 10A are graphs of target pressure amplitude distributions at 10 mm, 30 mm and 45 mm distance, respectively, from a source of ultrasound. FIGS. 8B, 9B and 10B are graphs of simulated pressure amplitude distributions at the same distances of 10 mm, 30 mm and 45 mm, respectively, from a source of ultrasound. In some embodiments, the simulated pressure distribution shown in FIGS. 8B, 9B and 10B is obtainable using a customizable lens designed using, for example, IASA-based methods. The sample targets in FIGS. 8A, 9A and 10A may correspond to stones, calcifications, concretions, blood vessels, tumors, etc., that are treated by the ultrasound.

In some embodiments, a time-varying signal alters the beam pattern of a single-lensed transducer. For example, a customizable lens may generate multiple patterns at different frequencies simultaneously or may generate a single pressure pattern for a finite temporal period. In one embodiment, the frequency of a sinusoidal ultrasound signal may be varied over time to change the pattern, either continuously as a frequency chirp, or discretely in intervals. In another embodiment, a short signal pulse may be generated by the transducer to produce a temporary holographic image for a therapy such as shock wave lithotripsy, burst wave lithotripsy, or histotripsy. In other embodiments, the customizable lens may be designed to produce a target distribution of ultrasound phases that, for example, push the target in a desired direction.

Comparison of FIGS. 8A, 9A and 10A with their counterparts in FIGS. 8B, 9B and 10B indicates that the match between the target and simulated pressure amplitude distribution is good in view of the intricacy of the features of the target distribution and difficulty of generating different target patterns at different focal distances.

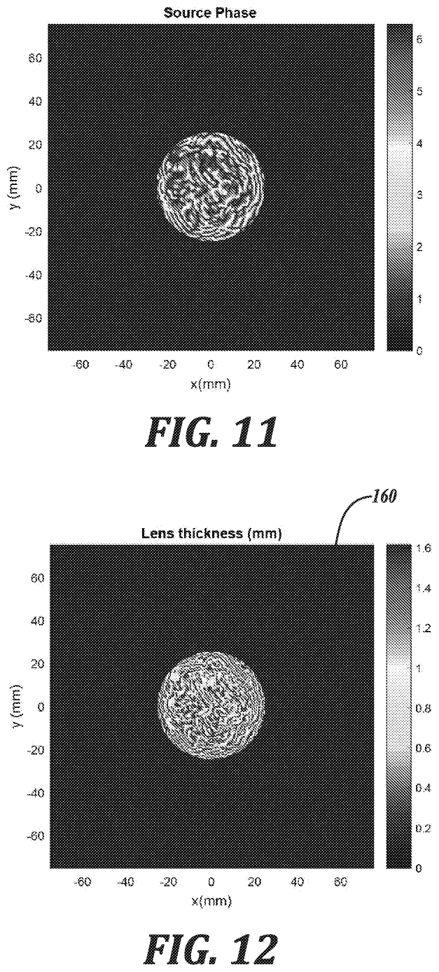

FIG. 11 is a graph of source phase for a customizable lens in accordance with an embodiment of the present technology, and FIG. 12 is a graph of a lens thickness for the customizable lens of FIG. 11. Thickens of the customizable lens 160 may be determined with the above-described IASA method. This map of thickness and the corresponding map of lens curvature define the customizable lens 160, which is a diffractive lens that may be produced by 3D printing. As explained above, when combined with a transducer that generates ultrasound at prescribed amplitude and frequency, the customizable lens produces a series of pressure amplitude distributions described in FIGS. 8B, 9B and 10B.

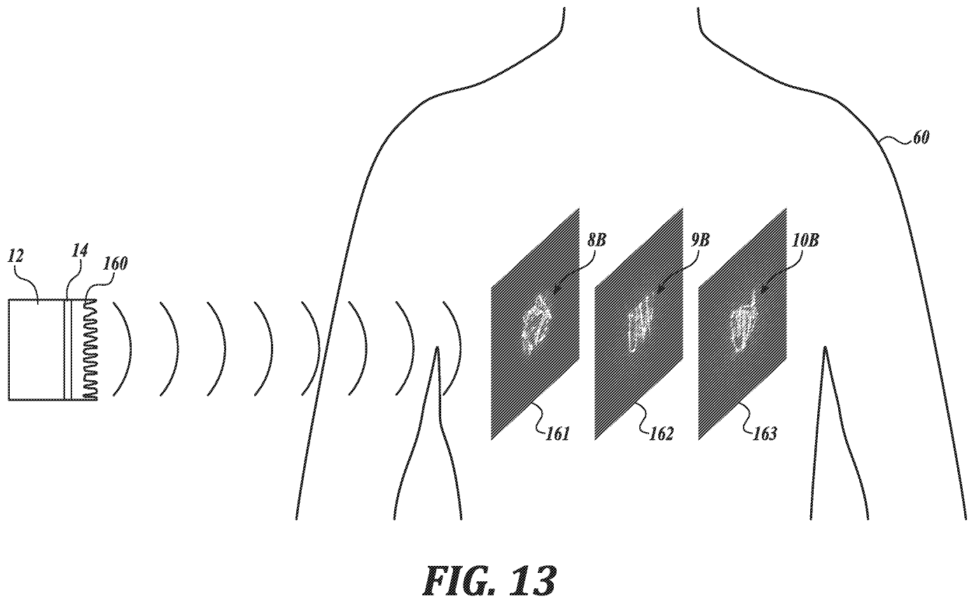

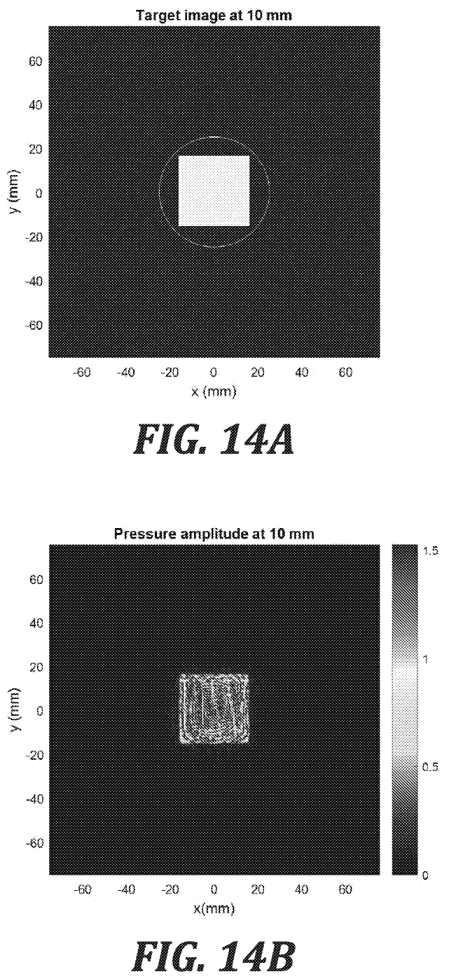

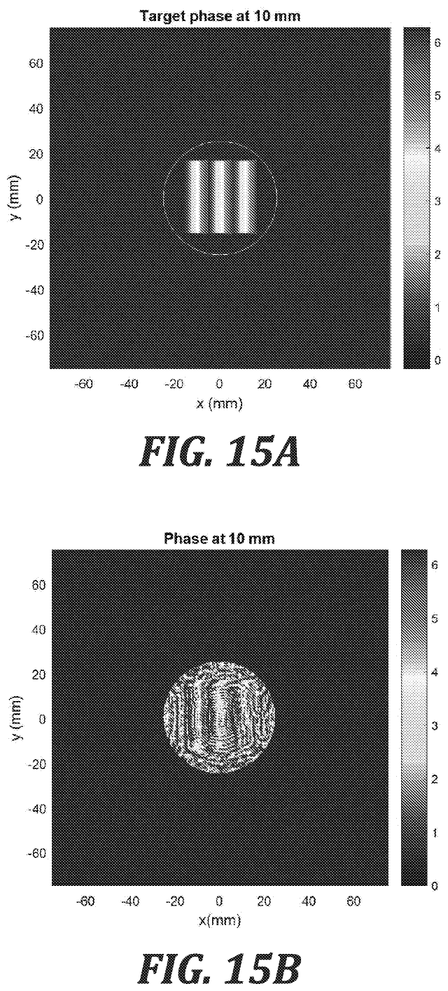

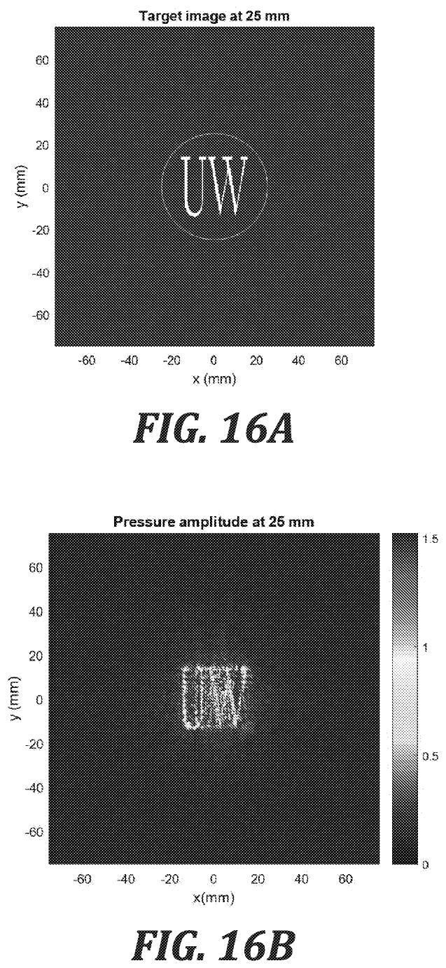

FIG. 13 is a schematic diagram of using the customizable lens in accordance with an embodiment of the present technology. In operation, the customizable lens 160 is attached to the ultrasound transducer 12 via the interface 14. When the ultrasound transducer 12 generates waves at the prescribed frequency and amplitude, the ultrasound beams combine to produce pressure fields shown in images 161-163. In practical operation, the images 161-163 correspond to the treatment areas in a body 60. In different embodiments, the ultrasound phase field may be controlled separately or in conjunction with the amplitude field, as explained below in conjunction with FIGS. 14A-16B.

FIGS. 14A, 15A and 16A are graphs of target distributions of pressure amplitude at 10 mm, phase at 10 mm, and pressure amplitude image at 25 mm, respectively. Correspondingly, FIGS. 14B, 15B and 16B are graphs of simulated pressure amplitude at 10 mm, simulated pressure phase at 10 mm, and simulated pressure amplitude image at 25 mm, respectively. The distances are measured from the source of ultrasound.

In some embodiments, the simulated pressure amplitude and phase distributions are obtainable using a single lens designed by the IASA-based methods. In general, the match is good between the target and simulated distribution for both the amplitudes and phases, and at both distances of interest. Therefore, it is possible to obtain different distributions of different parameters (e.g., pressure amplitude and phase) at different target distances from the source of ultrasound.

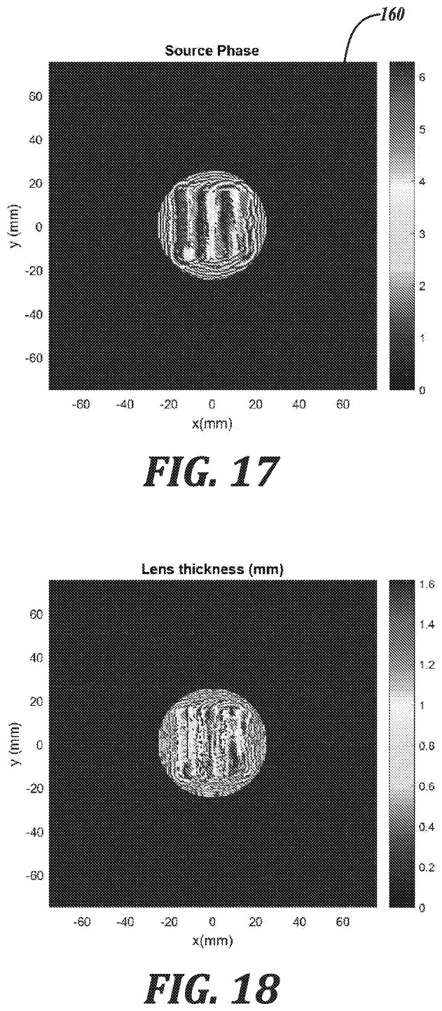

As explained above, the customizable lens can be designed based on the target amplitude/phase distributions that are shown in FIGS. 14A, 15A and 16A. Attributes of such customizable lenses are shown in FIGS. 17 and 18. FIG. 17 is a graph of source phase for a customizable lens in accordance with an embodiment of the present technology, and FIG. 18 is a graph of a lens thickness for the customizable lens. As explained above, the map of customizable lens thickness and/or curvature can be used to manufacture the customizable lens 160, using, for example, additive manufacturing methods like 3D printing.

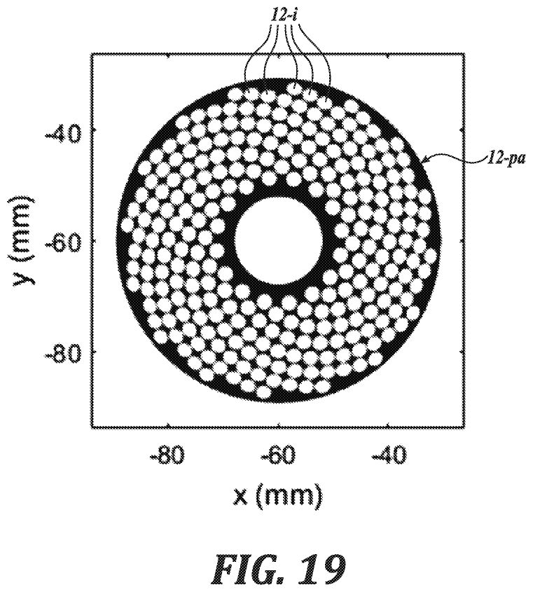

In some embodiments, the transducer may be a phased array having transducer elements that are electronically controlled to generate the amplitude and/or phase at proper frequency. FIG. 19 is a schematic diagram of a phased array 12-pa in accordance with an embodiment of the present technology. The spatial resolution of the phased array elements 12-i is limited to the size of the elements. As a result, the phased array 12-pa may not be able to produce an image with the same fidelity as a transducer with a customizable lens. However, using the phased array source of ultrasound, a target distribution of pressure amplitude, phase, etc., may be changed relatively rapidly. In some embodiments, the above-described IASA method may be used to design a distribution and spacing of the phased array elements 12-i.

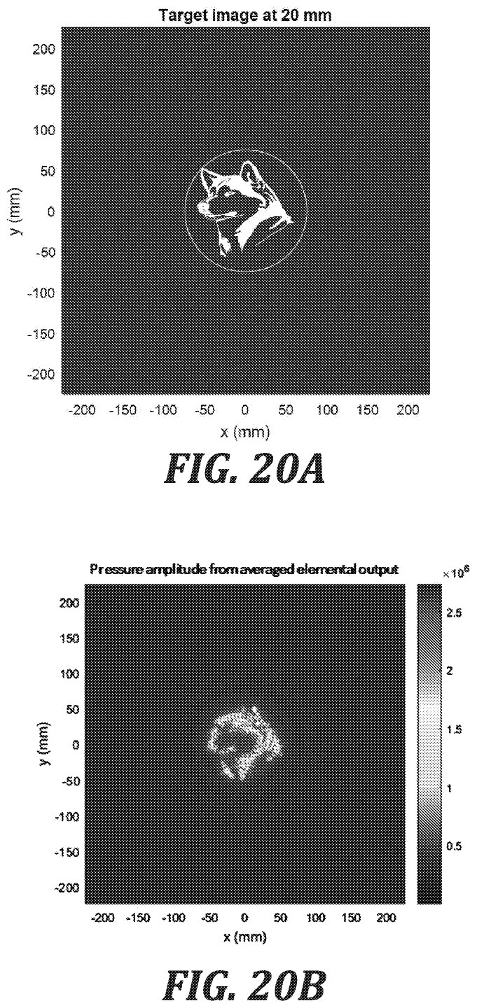

The illustrated phased array elements 12-i are arranged in a plane, but, in other embodiments, the elements 12-i may be arranged along a curved surface. For example, the phased array elements 12-i may be angled towards a focus, and may be activated at the phase that takes into account this spatial distribution of the phased array elements 12-i. In some embodiments, such a transducer is more efficient than a planar transducer with a lens. Illustrative results obtained with a phased array are discussed with reference to FIGS. 20A-20B below.

FIG. 20A is a graph of a target pressure field in accordance with embodiments of the present technology. The target pressure field is defined as a binary image (bright areas corresponding to high pressure, and dark areas corresponding to low pressure) at 20 mm focal distance. The white circle shows the outline of the transducer aperture.

FIG. 20B is a graph of pressure amplitudes obtained by the phased array 12-pa. In operation, the phase, amplitude and/or frequency of the phased array elements 12-i is controlled to approximate target distribution of pressure amplitudes at a required focal distance. The illustrated distribution of pressure amplitudes in FIG. 20B appears granular, and some details of the target image are not completely replicated (e.g., the ears of the Husky). However, the target image of FIG. 20A is relatively complex, and most of the practical targets 22 are less complex. In some embodiments, the phased array 12-pa may be used in conjunction with the customizable lens 160.

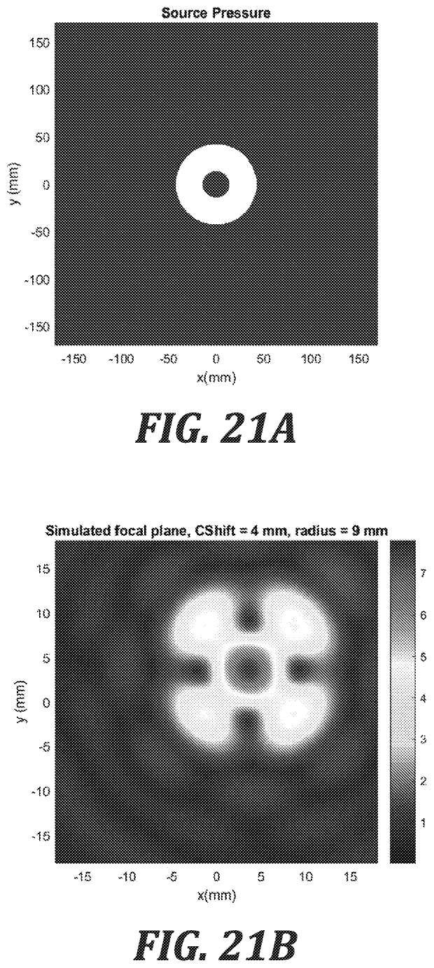

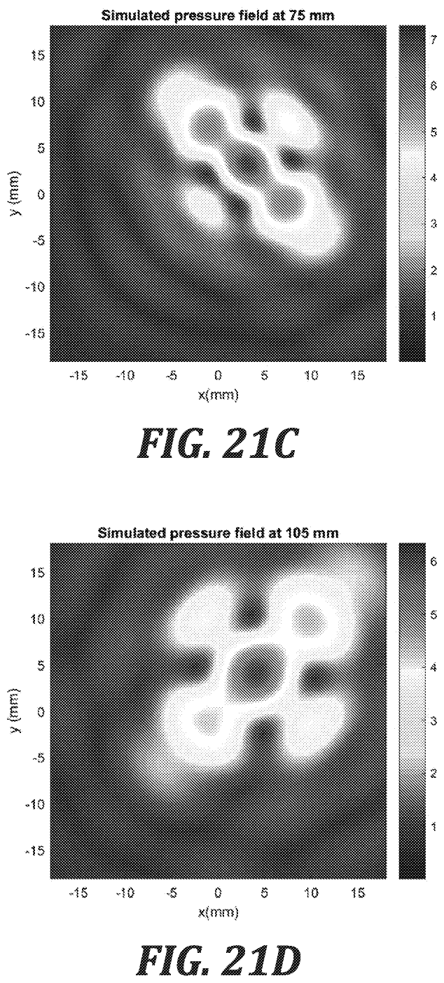

FIG. 21A is a graph of source pressure field, and FIGS. 21B-21D are graphs of the simulated pressure fields at different focal distances in accordance with embodiments of the present technology. In the illustrated embodiment, the target pressure field was offset from the centerline axis (Z-axis) of the transducer by 4 mm in X-axis and 4 mm in Y-axis. FIG. 21B shows the resulting pressure fields in a focal plane that is 90 mm away (in Z-direction) from the plane of the transducer. FIGS. 21C and 21D show the resulting pressure fields at 75 mm and 105 mm, which are respectively −15 mm and +15 mm from the plane of the transducer.

As shown in FIGS. 21B-21D, the asymmetry in the target field is achievable and a relatively large focal area is replicated at the prescribed 4 mm offset in the X-axis and Y-axis. The highest-pressure amplitude is located in the focal plane of 90 mm (FIG. 21B).

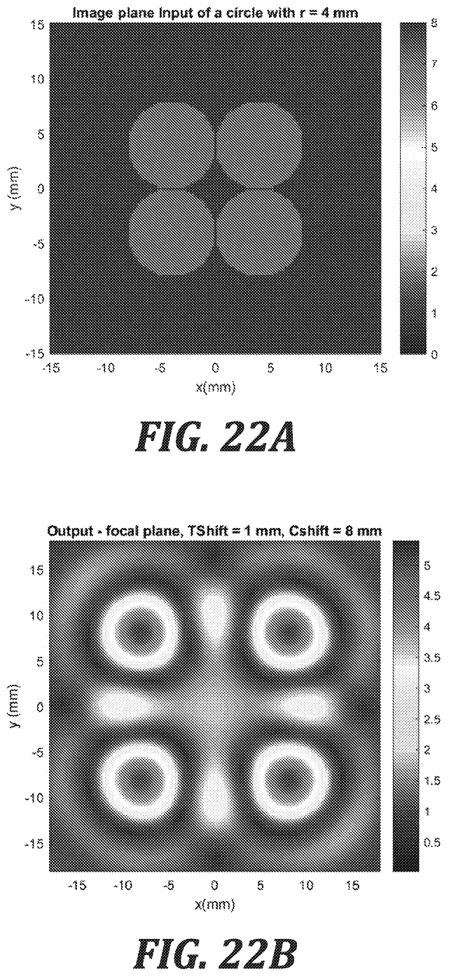

FIGS. 22A and 22B are graphs of sample pressure fields in accordance with embodiments of the present technology. FIG. 22A is a target pressure field that includes four circle-shaped areas of high pressure, each circle having a diameter of 6 mm, while the center-to-center distance between the neighbor circles is 8 mm. The source of the pressure field (i.e., the ultrasonic transducer) is the same as the one shown in FIG. 21A but is shifted 1 mm in the X-direction and 1 mm in the Y-direction to introduce asymmetry. In some embodiments of the inventive technology, the asymmetry of the source improves the ability of the IASA method to model a customizable lens that can generate complex fields like the one shown in FIG. 21A. FIG. 22B shows the pressure field distribution obtained in the target focal plane that is 90 mm away from the transducer along the Z-axis. The target pressure field corresponds well to the target field shown in FIG. 21A.

In general, many of the sample pressure or phase distributions shown in FIGS. 4-22B exceed the level of feature detail achievable with conventional ultrasound systems. The apparatuses and methods of the inventive technology achieve significantly more granular and precise targeting of the objects (e.g., stones, calcifications, blood vessels, tumors, etc.) in the body.

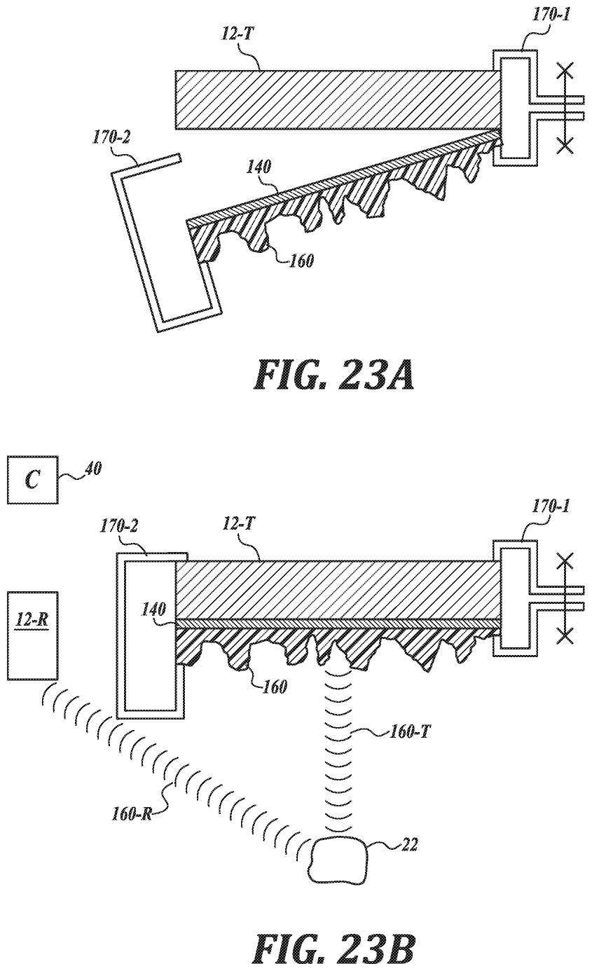

FIGS. 23A and 23B are schematic drawings of a lens holding mechanism in accordance with embodiments of the present technology. Some embodiments of the inventive technology include an interchangeable customizable lens 160 that is removeably mounted to the transmit transducer 12-T. In some embodiments, the customizable lens 160 is kept against the transducer 12-T by a holding mechanism 170 and a temporary interface 140. The temporary interface 140 may include a paste (e.g., a polymer, a rubber-like material, an epoxy, etc.) having acoustic properties selected to match that of the customizable lens 160. The temporary interface 140 may be mounted to the customizable lens 160 prior to mounting the customizable lens 160 to the transducer 12-T.

In some embodiments, the holding mechanism 170 is a quick-change clamp assembly, including a clamp 170-1, anchored to one location of the transducer 12-T, and a quick-change bracket 170-2, fixed to the customizable lens 160 at another location, for example, opposite to the location of the clamp 170-1. In some embodiments, the clamp 170-1 is attached to the customizable lens 160. The clamp 170-1 may include a recess or a receptacle for inserting one end of the customizable lens 160, after the temporary interface 140 has been mounted to the customizable lens 160. Once aligned, the quick-change 170-2 may fit the customizable lens 160 and temporary interface 140 conformably to the surface of the transducer 12-T.

In some embodiments, the holding mechanism 170 is a threaded retaining ring that reversibly mounts the customizable lens 160 and the temporary interface 140 to the transducer 12-T using a threaded junction. In some embodiments, the holding mechanism 170 includes a plurality of clip-in fasteners attached in part to the customizable lens 160.

FIG. 23B shows the customizable lens 160 mounted to the transducer 12-T. In operation, the system may acquire reflected ultrasound waves by a receiver 12-R. The reflected sound waves may be processed to show the shape, size and/or location of the target 22 as it undergoes the ultrasound treatment by the transducer 12-T, for example.

The receive transducer 12-R may be placed at an oblique angle relative to the direction of the propagating wave front 160-T emitted by the transmit transducer 12-T. In some embodiments, the receive transducer 12-R converts ultrasound waves into an electronic signal and sends the signal to the controller 40 for further processing. In some embodiments, the receive transducer 12-R is a sensor such as an ultrasonic microphone, a laser interferometer, etc. In other embodiments, the receiver transducer 12-R may have similar structure as the transmit transducer 12-T, except for being configured to receive and process the ultrasound, and not to transmit the ultrasound. In some embodiments, the transducer 12-T may fulfil both transmit and receive functions.

In some embodiments, the controller 40 provides information about the condition and position of the target 22. For example, the propagating wave front 160-T may fragment or move the target 22, whereupon the receiver transducer 12-R may measure the change in the target 22 based on the reflected wave front 160-R, and then provide data to the controller 40.

In some embodiments, the receiver 12-R is, e.g., Computed Tomography (CT), magnetic resonance imaging (MRI) or other imaging system. Based on the ultrasound, CT, and/or MRI imaging, a 3D reconstruction of the stone may be obtained. In operation, the image may be used for designing the customizable lens and/or for monitoring the treatment process. In some embodiments, the customizable lens may be further modified by, for example, machining, based on the observed progress of the ultrasound treatment of the target 22.

Many embodiments of the technology described above may take the form of computer- or controller-executable instructions, including routines executed by a programmable computer or controller. Those skilled in the relevant art will appreciate that the technology can be practiced on computer/controller systems other than those shown and described above. The technology can be embodied in a special-purpose computer, controller or data processor that is specifically programmed, configured or constructed to perform one or more of the computer-executable instructions described above. Accordingly, the terms “computer” and “controller” as generally used herein refer to any data processor and can include Internet appliances and hand-held devices (including palm-top computers, wearable computers, cellular or mobile phones, multi-processor systems, processor-based or programmable consumer electronics, network computers, mini computers and the like).

From the foregoing, it will be appreciated that specific embodiments of the technology have been described herein for purposes of illustration, but that various modifications may be made without deviating from the disclosure. Moreover, while various advantages and features associated with certain embodiments have been described above in the context of those embodiments, other embodiments may also exhibit such advantages and/or features, and not all embodiments need necessarily exhibit such advantages and/or features to fall within the scope of the technology. Accordingly, the disclosure can encompass other embodiments not expressly shown or described herein.

1. An ultrasonic therapy system configured to apply ultrasound to a target in a body, comprising:
an ultrasonic transducer configured to generate the ultrasound; and
a customizable lens configured to direct the ultrasound onto an area of a target,
wherein the target is an object or a portion of the object in the body, and wherein the customizable lens is designed and produced based on a determined size, shape or location of at least one target being a design input for the customizable lens, wherein the customizable lens is designed by an iterative numerical computational method based on the at least one target such that a focal area of the customizable lens corresponds-to or exceeds a size and a shape of the object.

2. The system of claim 1, wherein the target is defined by at least one acquired image of the object in the body.

3. The system of claim 1, wherein the customizable lens is a three-dimensional (3D) printed lens.

4. The system of claim 1, wherein the iterative numerical computational method is based on an iterative angular spectrum approach (IASA).

5-6. (canceled)

7. The system of claim 1, wherein ultrasound pressure phases are focused onto the focal area.

8. The system of claim 3, wherein the customizable lens is configured to produce a plurality of target distributions of the ultrasound at a corresponding plurality of focal distances from the customizable lens.

9. The system of claim 3, wherein the customizable lens is configured to produce the plurality of target distributions of the ultrasound at a corresponding plurality of ultrasound frequencies.

10. The system of claim 2, wherein the at least one acquired image of the object is modified to introduce an asymmetry in a target ultrasound field of the focal area of the object.

11. The system of claim 10, wherein the customizable lens produces multiple high-pressure areas within the target ultrasound field.

12. The system of claim 1, further comprising:
a mechanism configured to mate the customizable lens with the ultrasonic transducer; and
an interface material configured to temporarily attach the customizable lens with the ultrasonic transducer.

13. The system of claim 12, wherein the mechanism is selected from a group consisting of a quick-change clamp, a hinge and a bolt.

14. The system of claim 1, wherein the ultrasound transducer is a phased array transducer comprising a plurality of ultrasound sources.

15. The system of claim 1, wherein the plurality of ultrasound sources of the phased array transducer is arranged along a curved surface.

16. A method for applying an ultrasound to a target in a body, comprising:
defining a customizable lens based on a determined size, shape or location of at least one target, wherein the customizable lens is designed by an iterative numerical computational method, and wherein the target is an object or a portion of the object in the body;
generating the ultrasound by an ultrasonic transducer; and
directing the ultrasound onto an area of the object by the customizable lens such that a focal area of the customizable lens corresponds to or exceeds a size and a shape of the object.

17. The method of claim 16, further comprising:
acquiring an image of the object, wherein the iterative numerical computational method is based on an iterative angular spectrum approach (IASA).

18. The method of claim 17, further comprising:
acquiring additional images of the object while the target is being treated; and
based on acquiring the additional images of the object, modifying the customizable lens.

19. The method of claim 16, further comprising manufacturing the customizable lens by three-dimensional (3D) additive-printing.

20. The method of claim 16, further comprising:
applying an interface material to a surface of the customizable lens;
mating the customizable lens with the ultrasonic transducer via the interface material; and
after directing the ultrasound onto the area of the body, removing the customizable lens from the ultrasonic transducer.

21. The method of claim 16, wherein the ultrasound transducer is a phased array transducer comprising a plurality of ultrasound sources.

22. The method of claim 16, wherein directing the ultrasound onto the area of the body includes focusing ultrasound pressure amplitude distribution or ultrasound pressure phase distribution over the focal area.

23. The method of claim 16, further comprising:
prior to defining the customizable lens, introducing an asymmetry in a target ultrasound field of the focal area; and
in response to introducing the asymmetry, generating multiple high-pressure areas within the target ultrasound field by directing the ultrasound onto the focal area by the customizable lens.

24. The method of claim 16, further comprising generating a plurality of target distributions of the ultrasound at a corresponding plurality of focal distances from the customizable lens.

25. A non-transitory computer readable medium having computer executable instructions stored thereon that, in response to execution by one or more processors of one or more computing devices, cause the one or more computing device to perform actions comprising:
acquiring an image of a size, shape or location of an object in a body; and
determining a shape of a customizable lens based on the acquired image of the object in the body, wherein the customizable lens is designed by an iterative numerical computational method,
wherein the customizable lens is configured for mating with an ultrasound transducer, and wherein the customizable lens is configured to direct the ultrasound transducer onto an area at the object such that a focal area of the customizable lens corresponds-to or exceeds a size and a shape of the object.
